(19) United States
(12) Patent Application Publication
(10) Pub. No.: US 2003/0113916 A1
(43) Pub. Date: Jun. 19, 2003

(54) Method for producing somatic embryos of scot pine (p sylvestris)

(76) Inventor: Carlos Ramirez Serrano, Jalisco (MX)

Correspondence Address: Davis Wright Tremaine, 2600 Century Square, 1501 Forth Avenue, Seattle, WA, 98101-1688, US

(21) Appl. No.: 10/220,483

(22) PCT Filed: Feb. 28, 2001
(86) PCT No.: PCT/MX01/00013

(30) Foreign Application Priority Data
Feb. 29, 2000 (MX) 002107

(51) Int. Cl. C12N 5/00; C12N 5/02; A01H 7/00

(52) U.S. Cl. 435/422; 435/430; 800/319

(57) ABSTRACT

A method for producing mature somatic embryos, such method was improved with cryopreserved genotypes and it was applied successfully in a wide range of genotypes and families This method comprises the establishment and continuing proliferation of at least 40% of induced genotypes, treatment for reducing proliferation rates, treatment for initiating the development of immature somatic embryo, somatic embryo maturation of at least 80% of the treated genotypes belonging at least 90% of the tested families. Such result were achieved by simultaneously application of different ammonium to nitrate molar rations depending on the step which are included in the present method.

### FIELD OF THE INVENTION

The invention refers a new meted to carry out the gymnosperms somatic embryogenesis process that is also efficient for cryopreserved genotypes. The method allows plant regeneration of a wide range of genotypes and families. For each step the key is to use at the same time several media supplemented by different ammonium to nitrate molar ratios, this strategy allows the establishment and proliferation of somatic embryos. The embryogenic tissue is established and give rise continue proliferation by transferring the tissue from one medium to at least two mediums supplemented each one with different ammonium to nitrate molar ratios. However for the development of somatic embryos the low ammonium to nitrate molar ratio produces mature somatic embryos up to 80% of the genotypes belonging to 90% of the families tested.

### BACKGROUND OF THE INVENTION

Conifer propagation by in vitro cultures is increasing its importance in order to provide enough material for the demanding of forest products. The seedling propagation is the traditional way to produce large stock of plants for reforestation, mainly for conifer trees. However, intrinsic restrictions avoid the use of sexual reproduction as large scale of seed production and best quality seedlings. Which have caused extensive interest to developed and improve methods for asexual reproduction; especially economically important conifers such Pinus, Pseudotsuga and Picea. Asexual propagation through selection techniques provides high genetic gains by cloning desirable progeny to perform homogeneity and superior growth. Such type of plants is used for reforestation purposes.

Propagation by somatic embryogenesis means the methods whereby embryos are produced in vitro from plant tissue or single cells. The embryos are called as somatic embryos because they are ensuing from vegetative plant tissue rather than from sexual reproduction. Somatic embryogenesis propagation allows capturing all genetic gains and large-scale plant production (Gupta et al., 1993). Furthermore, somaclonal variation (done by in vitro cultures) among subclones belonging to one genotype was not significant, which means that massive propagation of a desirable genotype can be carried out with high genetic stability (Eastman et al., 1991). Conifer somatic embryogenesis is also used to produce large amount of synthetic seeds for low seed production species; for cloning varieties resistant to pesticides, diseases and environmental stress; it is also considered an alternative method for endangered and rare species conservation and also for propagation of ornamental varieties (Attree and Fowke, 1993).

For a long 15 years the conifer regeneration protocols have been improved since the first report of these types of plants were reported by Hakman and von Arnold (1985). However, only few protocols assure massive propagation. The most economically important conifer species are Picea and Pinus. There are 30 Picea species mainly distributed to cooler areas of boreal hemisphere. Pine trees belong to the most important conifer genera with around 140 species scattered around the northern hemisphere, whereas one of the pine diversity region is Mexico with around 60 species (McVaugh, 1992).

The result achieved on conifer somatic embryogenesis shows remarkable difference between Picea and Pinus species from initiation or induction of embryogenic tissue to regeneration of plants. Besides wide range of pine species has been consistently recalcitrant. Among Picea species the initiation rates are around 95% form immature zygotic embryos, and 55% from mature zygotic embryos (Tautorus et al., 1991). It is known that media compound is decisive, such the relation of inorganic nitrogen sources and the type of carbon source are the key for high initiation rates of somatic embryos (von Arnold, 1987). More over, there are quite numerous reports over high yield of mature somatic embryos, even produced by bioreactor process (Attree et al., 1994); however, only few reports have shown the plant regeneration and even less reports the adaptation in nursery conditions. In 1990 Webster and coworkers, have established more than 80% of somatic embryo plants under nursery condition belonging to 71 genotypes. Recently Hogberg et al, (1998) reported the adaptation of 25% of 2519 mature somatic embryos of Picea abies originated from 12 families. There are successful regeneration reports over of Picea species; such protocols are used for mass propagation of selected material.

Actually the improvements achieved on pine somatic embryogenesis have shown equal or superior results as Picea and Pseudoisuga reports. Early the response to regeneration protocols have had limited response of few genotypes. Gupta y Durzan (1987a) reported regeneration of Pinus taeda, however it was merely a single plant (according to Pullman and Gupta, 1991). Klimaszewska and Smith (1997) have reported the success of regeneration of Pinus strobus somatic embryos; there were no given data of the number of them. Recently, Garín and collaborators (1998) have shown the response of 52 genotypes belonging to 13 families of the same specie, 800 mature somatic embryos from 30 genotypes in 12 families were obtained, with a conversion into plants of only 31% Lelu et al, (1999) have been shown the achievement of regeneration on. Pinus sylvesiris and Pinus pinaster; 48% of 360 mature somatic embryos from three genotypes, and 29% of 142 mature somatic embryos from three genotypes, respectively. To date the only method for pine trees that assure the regeneration of a wide range of genotypes and families was reported by Ramirez-Serrano and collaborators (Ramirez-Serrano et al, 1999a, 1999b), through which the production of thousand of somatic embryos from 70 out of 82 genotypes belonging to 19 out of 20 families, were obtained. These results mean that the pine somatic embryogenesis technology can be used for pine breeding and large-scale propagation. The full process was done on modified solid medium with various ammonium to nitrate molar ratios depending on the step.

Over the preservation of embryogenic potential, cryopreservation methods are the most useful tool for preserving embryos, cells and tissues of several species. However, it has been proved that crypreservation of conifer somatic embryos produces somaclonal variation and, consequently, the best genotypes could be altered in the maturation and germination process, although variation was only detected on embryogenic cultures, with no effect on regenerated plants (De Verno et al, 1999). In addition, protoplast derived Picea glauca plants, such protoplast were obtained from cryopreserved embryos. That demonstrate totipotency of plant cells was preserved (Attree et al., 1989). A different technique to maintain the embryogenic capacity for one year is to put small pieces of embryogenic tissue on solid medium onto Erlenmeyer flasks covered by cerum caps (Joy et al., 1991). Chilling treatment of immature somatic embryos assures the maintenance of embryogenic capacity, prior to arise proliferation and maturation (Ramirez-Serrano, 2000).

High improvement has been obtained through suspension cultures in liquid medium, in case of Picea and Pseudotsuga are efficient routines for both species. The somatic embryos proliferate faster, and costs are lower (Aitken-Christie and Connett, 1992; Gupta et al., 1993). However, most of pine species are recalcitrant to suspension cultures (Handley III, 1996). There are few references on pine suspension cultures: Pinus taeda (Gupta and Durzan, 1987a, 1987b; Gupta and Pullman, 1991; Pullman and Gupta, 1991), Pinus strobus (Finer et al. 1989), Pinus caribaea (Laine and David, 1990; Laine et al 1992) and Pinus maximartinezii (Ramirez-Serrano 1996). Over the last specie was reported 2 25% of embryogenic tissue induced on immature gametophytes, in a total of 18 genotypes, where only 8 genotypes were directly established in liquid medium (without treatment in solid medium), proliferated from 50 to 700-1500 immature embryos per ml, in 7 or 15 days between subcultures, depending on the genotype However, only abnormal embryos were produced (Ramirez-Serrano, 1996) At this time pine mature somatic embryos arise from suspension cultures that were preserved by chilling treatment (Ramirez-Serrano, 2000).

The maturation process is usually started by activated charcoal pretreatment in order to enhance response to maturation medium, by absorbing inhibitors in the medium, such as ethylene and plant growth regulators (George, 1993). The basal solidified medium used is the same as in initiation and proliferation steps, supplemented with a carbon source, a razemic abscisic acid (ABA), and a desiccant compound, such polyethylene glycol (PEG) or high sugar's concentration in order to increase osmotic potential (Attree and Fowke, 1993) Dunstan and collaborators (1993) recommend ABA that synchronize the embryo development and increase the rates of mature somatic embryos, as well germination. Another compound that enhances pine somatic embryo's maturation is gellan gum at 1% in the maturation medium, without PEG (Klimazsewska and Smith, 1997; Lelu et al, 1999).

Before this method the following patents are hereby incorporated by reference: filed patent MX001185 wherein was used various ammonium to nitrate molar ratios to maintain suspension cultures of embryos which were preserved in freezer, and the maturation was acquired by the mixture of low ammonium to nitrate molar ratio, carbon source and desiccant agent other than PEG The U.S. Pat. No. 5,534,434 refers a new media compounds specific for suspension cultures of Pinus taeda, whereas the nitrogen molar ratios are different to this proposal. The U.S. Pat. No. 5,563,061 restrict maltose utilization for proliferation cultures on solid medium only; the present method addresses that proliferation is not depending on maltose compound. Over the maturation media composition for conifer somatic embryos, the following patents limit the key compound or mixtures. U.S. Pat. No. 5,034,326 restrict the mixture of abscisic acid (ABA) and activated carbon (AC) in the maturation medium. U.S. Pat. No. 5,036,007 constrain the mixture of polyethylene glycol (PEG), ABA and AC, to enhance maturation response by gradual attenuation of ABA concentration. U.S. Pat. No. 5,187,092 limit the mixture of ABA and carbon source. U.S. Pat. No. 5,236,841 protect the gradual ABA diminution and the increment of desiccant compound. U.S. Pat. No. 5,294,549 restrict the mixture of ABA, AC and giberelic acid. U.S. Pat. No. 5,413,930 protect the mixture of ABA, gelling agent and carbon source. U.S. Pat. No. 5,491,090 refers as it invention the combination of gelling agent, carbon source, AC and ABA. U.S. Pat. No. 5,731,203 take care of the combination of gelling agent, carbon source and ABA. U.S. Pat. No. 5,731,204 restrict the mixture of PEG, AC, ABA and carbon source. U.S. Pat. No. 5,856,191 constrain the mixture of ABA, gelling agent and carbon source. S05985667 patent protect the mixture of ABA, PEG and carbon source. WO9963805A2 patent take care of the increasing levels of plant growth regulators (ABA) and/or desiccant compound.

There are several differences between the patents mentioned and this method in order to produce mature somatic embryos. The most important is the potential to regenerate a wide range of genotypes and families, while the patented methods assure response from few genotypes. In order to achieve that potential, the strategy for establishment and proliferation of embryogenic tissue was subculturing each genotype simultaneously in three culture media, each medium with a different ammonium to nitrate molar ratio. A new strategy was the reduction of proliferation levels in medium with low ammonium to nitrate molar ratio followed by treatment to block the proliferation with high level of nitrate and activated charcoal in order to encourage the response to maturation medium; such maturation medium has as key factor the low ammonium to nitrate is relation (10:90), carbon source, maturation promoter and PEG as desiccant compound. This method arises the somatic embryo maturation of a wide range of genotypes tested belonging to almost all families evaluated.

The main object was to achieve a pine regeneration method for a wide range of genotypes and families, in order to assure the production of thousand of somatic mature embryos, and later their establishment in soil.

Another object is to establish a useful method for breeding programs wherein the possibility to regenerate each selected elite tree is available.

### SUMMARY OF THE INVENTION

According with the present invention, a new meted to achieve plant regeneration from a wide range of genotypes cryopreserved or not. This method includes the establishment and permanent proliferation for a wide range of genotypes, having a reduction of proliferation levels, applied a pretreatment to initiate embryo development and maturation treatment. All process is given by the utilization of media modification with one or various ratios of inorganic nitrogen source, depending on the particular step of the somatic embryogenesis process giving to gymnosperms. The induction process in not included in this method, such step was arisen by known techniques already established for gymnosperms trees.

In this method for gymnosperm somatic embryogenesis, the term called as “basal modifications” is the simultaneous employment of various ammonium to nitrate molar ratios in order to subculture the embryogenic tissue as follows: 80:20, 40:60, and 10:90. Such relations allow the establishment of embryogenic tissue and proliferation of cryopreserved genotypes or no cryopreserved genotypes.

One of the keys of the present invention addresses the utilization of at least three mediums supplemented each one with a different ammonium to nitrate ratios (80 20, 40:60 and 10:90) during proliferation step, exchanging a small part of embryogenic tissue among three different media, such procedure allowed to achieve excellent proliferation of each genotype.

The invention comprises also a reduction of embryogenic tissue proliferation rates for a long 30 days as minimum, that is acquired through subculturing in a medium supplemented with low ammonium to nitrate molar ratio; high average of genotypes tested shown the reduction of proliferation rates treated with the relation 10:90. Under this treatment is thereby encouraging a better action of ABA and/or analogues, assuming that in the immature embryos the nitrogen pathway changes in order to metabolize nitrate instead of ammonium and consequently the somatic embryos get maturation in the proper medium However, other treatment has to be done with medium supplemented with an adsorbent and ammonium to nitrate molar ratio 10:90, as long as genotype needs, in order to start the development of immature somatic embryo head, such is the signal for transferring developing embryos onto maturation medium. It is also included washing of 1 gr of embryogenic tissue in a 50 ml container at least 3 times with sterile distilled water or sterile liquid medium supplemented only with mineral compounds. The embryos must be transferred to filter paper as thin dispersed layer in order to avoid embryogenic masses accumulation and humidity onto fresh medium

The present invention also included maturation of embryos on medium supplemented with high nitrate molar ratio (10-90), a carbon source, a maturation promoter such ABA and or analogues and at least a desiccant compound. Maturation treatment depends on the time in which somatic embryos develop cotyledons and are ready for the dormancy period (not included in this method). Gymnosperm somatic embryos produced by the present invention include conifer somatic embryos.

The present invention has the advantage of give nce of quantity of mature somatic embryos per genotype, and promoting better performance, that means the improvement of germination capacity and plant growth. More over, the plant regeneration is 47% from all obtained embryos. This invention addresses that the genotypes characterized for producing aberrant embryos only, without hypocotyls, following this method can develop as normal embryos.

The present invention is a significant advance in conifer somatic embryogenesis research, especially for pinaceae trees, in focus on the regeneration of a wide range of genotypes in which is included cryopreserved genotypes. According with proved results each genotype has different capacity for the maturation medium, that means the quantity of somatic embryos (1 to 300 embryos/200 mg fresh weight), give the key to produce the plants needed per genotype in order to evaluate properly during genetic breeding programs. Also, with this method the regeneration rates is up than 80% of induced genotypes per family belonging to 95% of the mother trees that producing embryogenic tissue. Besides, there are no differences among genotypes on germination capacity and plant conversion. As this method can be applied to a wide range of genotypes, the base for genetic transformation has been done. More over, with this method gives the opportunity to observe the development from embryo to plant, is now possible to study different studies as molecular, anatomical, physiological, genetics, etc

### FRIEF DESCRIPTION OF THE FIGURES

FIG. 1a represents the selection of media in order to establish and get continuing proliferation. In which is demonstrated each genotype has a different proliferation capacity and depending on ammonium to nitrate molar ratio (A′).

FIG. 1b represents genotypic influence on proliferation (A). It has been observed that the genotypes (G) with slow proliferation as well the treatment produces low proliferation rates, give rice the highest quantity of mature somatic embryos.

FIG. 2a represents the influence of both ammonium to nitrate molar ratio (A′) and type of carbon source in the maturation of somatic embryos (M). Wherein the genotype shows not significant giving influence over the quantity of mature somatic embryos, however that was misused when was established the best quantity of immature embryos per sample The embryogenic tissue used for this experiment was treated with the same ammonium to nitrate molar ratio as well in proliferation as in maturation step. Such results shown that reduction levels of proliferation rates and subculturing in medium supplemented with the relation 10:90 augment maturation levels of somatic embryos.

FIG. 2b shows high frequency of mature somatic embryos with cotyledons in the right development for desiccation process. The quantity and production time depending on the genotype, such time can be from 3 to 12 weeks. The maturation medium was supplemented with ammonium to nitrate 10:90, 3% maltose, 80 μM ABA, 7.5% PEG, and 0.35% gellan gum as gelling agent.

FIG. 3a shown germination in solid medium after desiccation treatment.

FIG. 3b shown the root development of pine plants.

FIG. 4a shown the response from 20 families (F) and their genotypes (G) for the followed process: proliferation (A), maturation (M) and germination (g). The genotypic response was from 1 to 300 mature embryos per 200 milligrams of embryogenic tissue utilized per sample, from 82% of tested genotypes

FIG. 4b shown pine plants under development after 4 months under ex vitro conditions.

### DETAILLED DESCRIPTION OF THE INVENTION

According with the present invention, is presented a new meted for producing gymnosperms mature somatic embryos from cryopreserved and no cryopreserved genotypes that was successfully tested for a wide range of genotypes and families. This method is focusing on giving treatments to immature somatic embryo parents of mature embryos. Such treatment is the establishment of embryogenic tissue in media with different basal modifications, having a continuing proliferation in at least three basal modifications, having a treatment in medium with low ammonium to nitrate molar ratio during a period of time as long as the proliferation is reduced to minimum level corresponding to each genotype. Having a second treatment with a chemical adsorbent in order to enhance somatic embryo development and the production of mature somatic embryos from most of tested genotypes in medium with high nitrate amount This method does not include induction of embryogenic tissue.

The present invention requires understanding and control of certain physiological factors that have an effect on induction, proliferation and maturation of somatic embryos, since it is known that stages of embryo development are similar in both zygotic and somatic embryos. As well as in vitro up take of inorganic nitrogen as key factor in each step of somatic embryogenesis in which a detailed description is required.

After fecundation, zygotic embryos of gymnosperms develop out of a non-nuclear structure, through a process that can have some variants. Later a structure develops within the archegonium with 16 elongated cells that will became a pre-embryo which itself may divide and give rise the normal cleavage polyembryogenesis of one or more genotypes when more than one egg is fertilized. In a normal process suspensor cells pushes the embryonic head towards the gametophyte and maturation starts when suspensor cells transfer nutriments to the embryo head from the gametophyte base. Simultaneously, desiccation starts in both zygotic embryo and gametophyte, in such a way that when the embryo has achieved complete maturation, moisture conditions are minimum for it to go into dormancy stage and stay in it until conditions are favorable for germination and normal growth into full plant. According to the above description, gymnosperm somatic embryogenesis requires the same conditions needed to produce zygotic embryogenesis. It is therefore assumed that requirements needed for induction, proliferation and maturation of somatic embryos are basically the same for all conifers species, with specific variants needed, according to the specie needs, in this case Pinus sylvestris. At the same time, regeneration methods developed for Gymnosperms are basically different from methods developed for Angiosperms

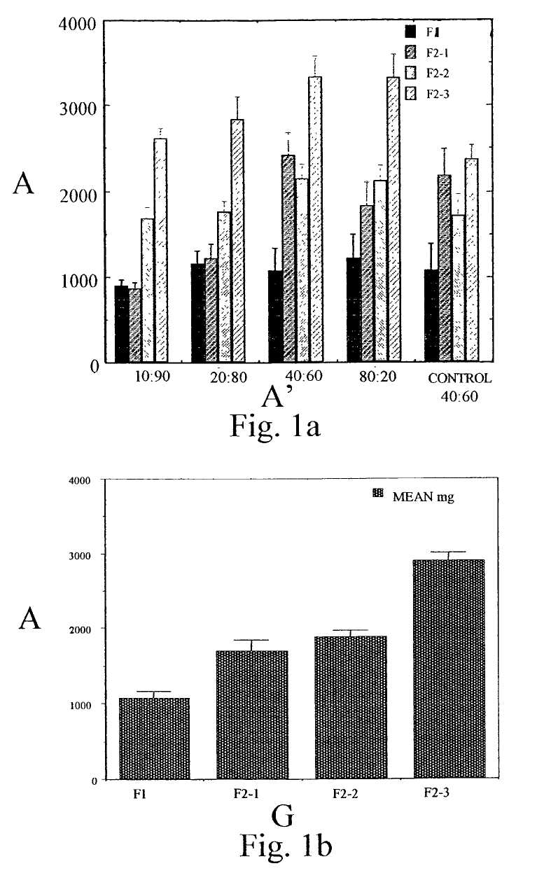

For some Pinaceas trees, as is here the case, the zygotic embryos utilized have to be under the first developing step, that means the pre-embryo is with only 16 cells, in order to promote the embryo cleavage as initiation of polyembryogenesis. It is known the media compounds are decisive to maintain proliferation, because of this, the effect of 4 ammonium to nitrate molar ratios and 2 plant growth regulator concentration over 4 genotypes were evaluated The result shown the proliferation is influenced by ammonium to nitrate molar ratio and genotype capacity (FIG. 1a); with non-effect by plant growth regulator concentration on proliferation rates (Table 3). The highest proliferation rate was obtained by the relation 40:60, however during time of evaluating data, the embryogenic tissue became partly dark, which is not good at this step. Because of that the ammonium to nitrate molar ratio 80:20 was chosen as superior treatment for induction step, wherein the embryogenic tissue was with excellent texture for a long the experiment evaluation. It must be emphasized that the four genotypes tested were cubcultured in a same medium previous to the experiment with various ammonium to nitrate molar ratios. In which were observed excellent translucent and mucilaginous embryogenic tissue after two subcultures without significant effect of ammonium to nitrate molar ratio. That means proliferation capacity is increasing and maintained by simple exchanging the embryogenic tissue from one medium to other different medium

TABLE 3
Variance analysis of four proliferating genotypes
(F1, F2-1, F2-2 y F2-3), ammonium/nitrate and plant
growth regulators (PGR) effect.

| Source | DF | F | Prob > F |
|---|---|---|---|
| Genotype | 3 | 70.95 | <0.0001 |
| PGR | 1 | 2.40 | 0.123 |
| NH₄/NO₃ | 4 | 8.59 | <0.0001 |
| Error | 151 | | |
| Total | 159 | | |

PGR: 3.5 mg/l 2,4-D + 0.5 mg/l BA and 2 mg de 2,4-D + 1 mg/l BA
Ammonium/nitrate molar ratio: 10:90, 20:80, 40:60 y 80:20 and DCR.

The treatment wherein was observed continuing proliferation without necrotic areas into embryogenic tissue was the relation 20:80 plus 3.5 mg/l 2,4-D and 5 mg/l BA, although the treatment wherein the high proliferation rates was 40:60 (FIG. 1a). It is known that a high average of embryogenic tissue stops proliferation as a result of genotypic effect encouraged by incorrect medium compounds, in order to avoid such effect the medium compounds were changed after two subcultures by a different ammonium to nitrate molar ratio (80:20, 40:60 and 10:90 respectively). Subculturing was done every 3 weeks. Table 4 shows the results achieved by this innovation.

TABLE 4
Number of established genotypes.

| Family | Initiated | Non-established | Established |
|---|---|---|---|
| 1 | 23 | 17 | 6 |
| 2 | 0 | 0 | 0 |
| 3 | 2 | 1 | 1 |
| 4 | 13 | 4 | 9 |
| 5 | 2 | 2 | 0 |
| 6 | 8 | 5 | 3 |
| 7 | 14 | 4 | 10 |
| 8 | 0 | 0 | 0 |
| 9 | 6 | 6 | 0 |
| 10 | 5 | 3 | 2 |
| 11 | 1 | 1 | 0 |
| 12 | 1 | 1 | 0 |
| 13 | 2 | 1 | 1 |
| 14 | 6 | 0 | 6 |
| 15 | 5 | 5 | 0 |
| 16 | 2 | 2 | 0 |
| 17 | 8 | 3 | 5 |
| 18 | 6 | 3 | 3 |
| 19 | 1 | 1 | 0 |
| 20 | 2 | 2 | 0 |
| 21 | 4 | 3 | 1 |
| 22 | 17 | 17 | 0 |
| 23 | 5 | 4 | 1 |
| 24 | 9 | 1 | 8 |
| 25 | 10 | 6 | 4 |
| 26 | 9 | 9 | 0 |
| 27 | 1 | 1 | 0 |
| 28 | 4 | 4 | 0 |
| 29 | 20 | 6 | 14 |
| 30 | 0 | 0 | 0 |
| 31 | 1 | 0 | 1 |
| 32 | 9 | 6 | 3 |
| 33 | 4 | 4 | 0 |
| 34 | 14 | 4 | 10 |
| 35 | 5 | 1 | 4 |
| 36 | 1 | 0 | 1 |
| | 220 | 127 | 93 |

According with these results, it is assuming the influence giving by genotype was decreased, as embryogenic masses of numerous genotypes and families were established through simultaneously mediums utilization with different ammonium to nitrate molar ratio.

The requirement for fast proliferation is high ammonium into the culture medium. In fact such compound is the nitrogen source mainly utilized by plants, whereas under in vitro conditions nitrogen alkaline source is required, consequently the ammonium to nitrate ratio has to be adjusted (FIG. 1a). More over, according with the statistical analysis the plant growth regulators not give significant effect over proliferation rates (Table 3), in which step was used 1 mg/l 2,4-D and 0 5 mg/l BA. The establishment is complicated as proliferation decline when is utilized a simple basal formulation. Under the light of that result, monitoring was done over the characteristics of embryogenic tissue arises from specific relation and later the effect done by transferring to new relation. With 80.20, after 5 subcultures embryogenic tissue from almost all genotypes tested, became rigid and white, but after transferring to 40.60 or 10.90 comes again to normal type as translucent and friable. Under treatment on the last relation the embryogenic tissue miss proliferation and became brown. Recovering of tissue is arisen by periodical transferring to other nitrogen relation as well proliferation. Such results seems to be caused by nitrogen up take changes, high proliferation rates with high ammonium amount, and when decreasing is done by nitrate when the pathway changes in order to up take nitrate instead of ammonium and consequently the lowest proliferation rates of immature embryos from all genotypes was acquired. (FIG. 1a)

Every genotype have positive response by transferring to new medium with different compound, in this case was the ammonium to nitrate molar ratio (Table 5, FIG. 1a), and proliferation have to be reduced in every genotype in order to enhance the response to maturation medium, which will be described later on. The innovative solution was done through simple transferring of some embryogenic masses from a medium to the other two media utilized at the same time in this step (80:20, 40:60 y 10:90)

By the previous description has been demonstrated that proliferation rates is influenced by ammonium to nitrate ratio and capacity of every genotype although they belonging to the same family (FIG. 1b). Continuing proliferation of all established genotypes was achieved for a long a year with no seems of bad type or null proliferation (Table 5).

TABLE 5
Proliferation achieved in 4 genotypes by ammonium/nitrate ratio.
Mean obtained after 4 subcultures starting with
100-180 mg of embryogenic tissue.

| NH₄/NO₃ | Replicas | Mean | Standard deviation |
|---|---|---|---|
| 10:90 | 32 | 1517.5 | 140.1 |
| 20:80 | 32 | 1744 6 | 150.7 |
| 40:60 | 32 | 2247.7 | 179.1 |
| 80:20 | 32 | 2123 5 | 179.0 |

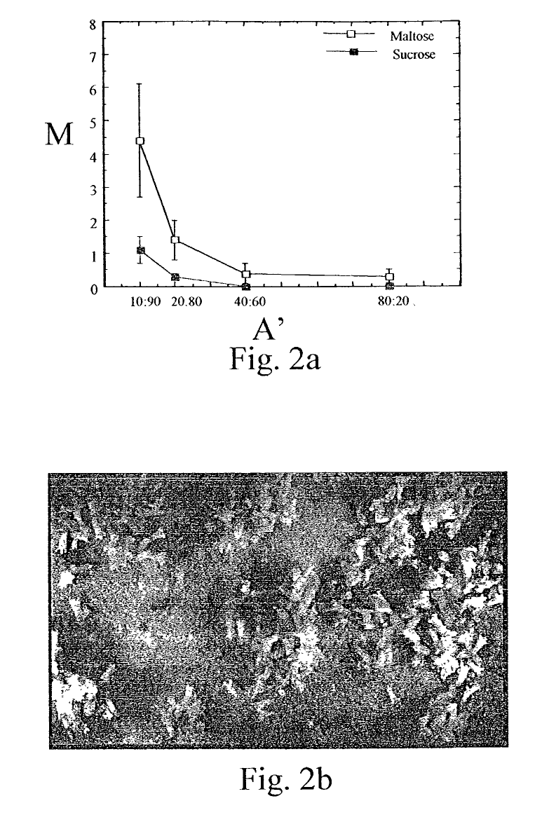

As it was mentioned early on, a very important sep was considered between proliferation and maturation processes, in order to grant access to maturation promoter achievement on immature embryo Such achievement is acquired by proliferation of immature embryos on the ammonium to nitrate molar ratios (Table 5, FIG. 1a). In despite of proliferation process where in the embryogenic masses take a brown light color meaning is the key to transfer to a medium with adsorbent in order to stop proliferation and/or encourage embryo development. For maturation process it was improved the method with four embryogenic cell lines previously cryopreserved, 2 of them (F1 y F2-3) where considered recalcitrant to maturation treatment and the other 2 (F2-1 y F2-2) only present aberrant embryos without hypocotyls; besides 4 ammonium to nitrate molar ratios, two carbon sources and all the known additives allowed the high production of matures somatic embryos were utilized, (FIG. 2b), suitable concentration of ABA, and desiccant compound. For maturation process is highly relevant the interaction between nitrogen and carbon source, and the type of the carbon source. In such way was evaluated 3% concentration of sucrose and maltose, with no effect over desiccant compound and gelling agent. High concentration of abscisic acid has to be added when starting maturation in order to acquire best quality embryos and avoiding precocious germination; high average of conifers protocols ABA concentration varying between 16 μM and 24 μM. However for Pinus genera ABA is required between 60 μM and 100 μM. Usually razemic mixture (t) is used as the best results achievement, under this evaluation only ABA 80 μM was applied, in the light on previous evaluation wherein were tested 20, 35 y 60 μM with non effect over the cell lines mentioned before. The orthodox embryos need a desiccation as requirement for normal germination. In order to produce gymnosperm mature somatic embryos with normal germination, is required such embryos developing on medium supplemented with high concentration of desiccant compound. Several sugars has been utilized as desiccant compounds in concentrations around 6 to 9%, however for this meted was applied a compound that is not up take by the cells, polyethylene glycol (PEG), this compound produces dry conditions enhancing the substance storage on the cells. PEG 4000 is the best supply as it produce a good viscosity The concentration used at the maturation step of this invention was 7.5% as most suitable, such concentration give proper condition to utilize only 0.35% gellan gum for gelling the medium. The mentioned gellan gum has high importance, as it is a polymer that not reacts with the rest of the medium compounds, giving support and physic consistence as requirement for in vitro culture medium The medium has to be solid with no liquefaction, and such polymer has to be not available for plant up take Also depending on its concentration the water up takes from the medium

Under the evaluation done to produce normal embryos, was utilized between 400 and 600 mg of embryogenic tissue per sample, such amount was dispersed on filter paper as no thin layer, with two washing only with liquid mineral medium and they were exposed only two weeks on medium supplemented with activated charcoal. According with statistical analysis the main effect to produce normal embryos is done by nitrogen relation and carbon source, in which all genotypes produced normal embryos, with no significant differences among genotypes by the quantity of embryos produced on this experiment (FIG. 2a, Table 6, Table 7). In the other hand, the production of abnormal embryos mainly depends on the genotype and ammonium to nitrate molar ratio with no effect of carbon source (Table 6, Table 7).

TABLE 6
Production of normal mature somatic embryos and abnormal embryos
depends on de media compounds (Genotypes F1, F2-1, F2-2, y F2-3).
The weight of embryogenic tissue utilized per sample was up of 400
mg, and such tissue was treated only 2 weeks in medium with
absorbent.

| Treatment | Sam- | Mature | Standard | Abnormal | Standard |
|---|---|---|---|---|---|
| NH₄/NO₃ | ples | embryos | deviation | embryos | deviation |
| 10:90 + Sucrose | 16 | 1.1 | 0.43 | 35.0 | 21.5 |
| 20.80 + Sucrose | 16 | 0.3 | 0.15 | 22.5 | 6.1 |
| 40:60 + Sucrose | 16 | 0.0 | 0.0 | 34.7 | 26.3 |
| 80:20 + Sucrose | 16 | 0.0 | 0.0 | 25.5 | 11.5 |
| 10:90 + Maltose | 16 | 2.7 | 1.7 | 54.0 | 8.6 |
| 20:80 + Maltose | 16 | 1.4 | 0.6 | 5.2 | 2.2 |
| 40:60 + Maltose | 16 | 0.4 | 0.2 | 16.0 | 12.2 |
| 80:20 + Maltose | 16 | 0.3 | 0.2 | 6.0 | 3.8 |

TABLE 7
Variance analysis (GLM) for the mine factors that give rice
maturation of different types of somatic embryos
(level of significance 99%).

| Source | DF | F | Prob > F |
|---|---|---|---|
| Normal embryo | | | |
| Genotype | 3 | 1.16 | 0.327 |
| Carbon source | 1 | 3.71 | 0 050 |
| NH₄/NO₃ | 4 | 3 05 | 0.019 |
| Error | 151 | | |
| Total | 159 | | |
| Abnormal embryos | | | |
| Genotype | 3 | 18.92 | <0 0001 |
| Carbon source | 1 | 0.00 | 0.990 |
| NH₄/NO₃ | 4 | 3.42 | 0 010 |
| Error | 151 | | |
| Total | 159 | | |

The quantity of embryos exposed to maturation medium was other key factor to improve the meted for producing somatic embryos, and such knowledge was giving by genotypes with slow proliferation, in which the small amount of embryogenic tissue produced, was exposed to maturation medium as embryo thin layer which in turn produced mature somatic embryos. In the other hand genotypes with high proliferation rates the response to maturation medium is practically nil.

In order to start maturation in which is included the maturation pretreatment in medium with 1% activated charcoal, ammonium to nitrate relation 10:90, 3% maltose and gelling with 0.35% gellan gum, have to be exposed between 150 to 200 mg per sample, as a thin layer is better. Previous has to be washed three times with distilled sterile water or with any liquid mineral medium to eliminate the substances induced proliferation, transferring to solid medium, any liquid has to be eliminated and the embryos and medium has to be exposed under sterile flow to avoid humidity. The pretreatment has to be applied until proliferation stops, that depending on the genotype (2-8 weeks) Under this procedure was enhanced the response and when was proved in 10 genotypes the achievement of mature somatic embryos was in 9 of them (Table 8 y FIG. 2b).

TABLE 8
Response of 10 genotypes to the medium supplemented with
ammonium to nitrate 10-90, 3% maltose 7.5% PEG and 0 35%
gellan gum treating between 150 to 200 mg of embryogenic
tissue per sample

| | | MATURE EMBRYOS | |
|---|---|---|---|
| Genotype | Samples | Mean per gfw | standard deviation |
| F1-1 | 3 | 383.5 | 72.6 |
| F2-1 | 3 | 73.5 | 28.9 |
| F3-1 | 3 | 10.0 | 10.0 |
| F4-1 | 3 | 256 6 | 49.8 |
| F5-2 | 3 | 53 0 | 14 2 |
| F5-3 | 3 | 253.5 | 133.8 |
| F10-1 | 3 | 15.0 | 15.0 |
| F15-4 | 3 | 105.0 | 40.0 |
| F15-8 | 3 | 53 5 | 13.0 |
| F20-1 | 3 | 0.0 | 0.0 |

gfw = gram fresh weight

The maturation method shows efficiency in which is included, reduction of proliferation in medium with low ammonium to nitrate 10:90, washing of immature embryos with distilled sterile water, amount of embryogenic tissue as only 150 to 200 treated per sample, spread as thin layer, pretreatment to start de embryo development as was specified, and exposition to maturation medium with ammonium to nitrate relation 10:90, 3% maltose, 80 μM ABA and 7.5% PEG4000 Efficiency was achieved in 82% of tested genotypes (Table 13). And according with Table 10, there are differences among families, genotypes and also in production time of mature embryos. By the results acquired from two families, variability among them can be observed over embryo production, different genotypic capacity, and the production time. Since 3 weeks until 12 weeks the mature somatic embryos were observed.

TABLE 9
Variance analysis (GLM) of 19 families, from 1 to 13 genotypes,
and three production times of cotyledonary embryos
(99% of significance)

| Source | DF | F | Prob > F |
|---|---|---|---|
| Family | 19 | 1.89 | 0.016 |
| Genotype | 12 | 2.06 | 0.021 |
| Production time | 2 | 5.38 | 0.005 |
| Error | 230 | | |
| Total | 263 | | |

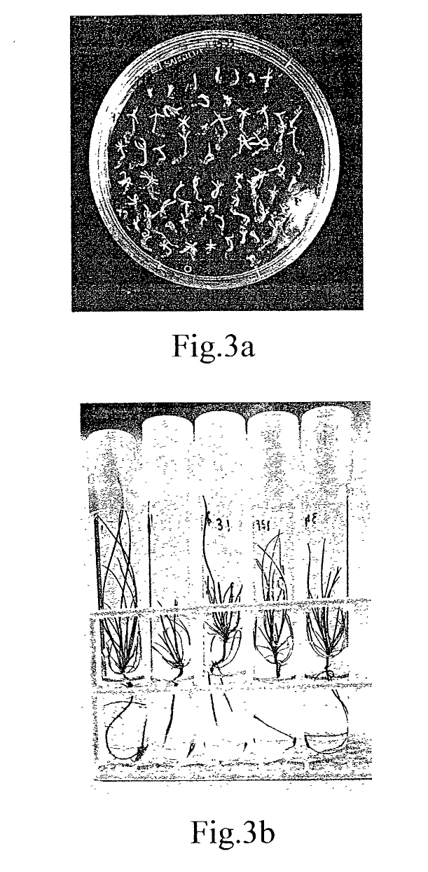

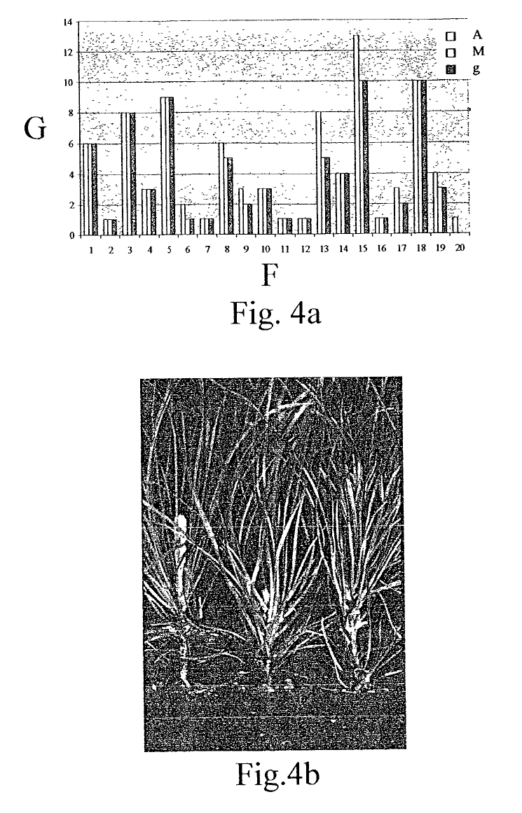

Based on the response of genotypes per family, it was found that high percent of established genotypes produce somatic embryos (FIG. 4a) This is the first report wherein were achieved mature somatic embryos from 82% of genotypes tested belonging to 95% of the families producing embryogenic tissue (Table 13). This method allows a high percent of germination and plant conversion (FIG. 3a). However is too important give a treatment to enhance better rooting (FIG. 3b) consecutively to establish under ex vitro conditions that are not included in this method (FIG. 4b).

TABLE 11
Family 5 response to the medium supplemented with ammonium to
nitrate molar ratio 10:90, 3% maltose, 7.5% PEG and 3.5%
gellan gum starting with 150-200 mg of embryogenic
tissue per sample.

| | | Taking times of | MATURE | Germin- | |
|---|---|---|---|---|---|
| Family | Genotype | mature embryo | embryos | ated | Plantlets |
| F5 | 1 | 1 | 5 | 5 | 2 |
| F5 | 1 | 2 | 13 | 11 | 7 |
| F5 | 1 | 3 | 90 | 74 | 50 |
| F5 | 2 | 1 | 0 | 0 | 0 |
| F5 | 2 | 2 | 0 | 0 | 0 |
| F5 | 2 | 3 | 1 | 1 | 1 |
| F5 | 3 | 1 | 12 | 12 | 10 |
| F5 | 3 | 2 | 27 | 10 | 6 |
| F5 | 3 | 3 | 4 | 1 | 0 |
| F5 | 4 | 1 | 55 | 53 | 37 |
| F5 | 4 | 2 | 410 | 299 | 248 |
| F5 | 4 | 3 | 508 | 311 | 287 |
| F5 | 5 | 1 | 6 | 2 | 1 |
| F5 | 5 | 2 | 20 | 19 | 5 |
| F5 | 5 | 3 | 19 | 14 | 4 |
| F5 | 6 | 1 | 0 | 0 | 0 |
| F5 | 6 | 2 | 2 | 2 | 0 |
| F5 | 6 | 3 | 0 | 0 | 0 |
| F5 | 7 | 1 | 39 | 30 | 30 |
| F5 | 7 | 2 | 52 | 49 | 49 |
| F5 | 7 | 3 | 80 | 79 | 79 |
| F5 | 8 | 1 | 8 | 7 | 5 |
| F5 | 8 | 2 | 40 | 20 | 19 |
| F5 | 8 | 3 | 40 | 30 | 29 |
| F5 | 9 | 1 | 0 | 0 | 0 |
| F5 | 9 | 2 | 28 | 20 | 0 |
| F5 | 9 | 3 | 45 | 40 | 0 |
| | | | 1504 | 1089 | 869 |

TABLE 12
Family 15 response to the medium supplemented with ammonium to
nitrate molar ratio 10.90, 3% maltose, 7.5% PEG and 3 5%
gellan gum starting with 150-200 mg of embryogenic
tissue per sample

| | | Taking times of | MATURE | Germin- | |
|---|---|---|---|---|---|
| Family | Genotype | mature embryo | embryos | ated | Plantlets |
| F15 | 1 | 1 | 11 | 9 | 0 |
| F15 | 1 | 2 | 20 | 6 | 0 |
| F15 | 1 | 3 | 12 | 3 | 0 |
| F15 | 2 | 1 | 2 | 2 | 1 |
| F15 | 2 | 2 | 4 | 1 | 1 |
| F15 | 2 | 3 | 4 | 1 | 1 |
| F15 | 3 | 1 | 18 | 9 | 9 |
| F15 | 3 | 2 | 8 | 6 | 3 |
| F15 | 3 | 3 | 2 | 2 | 1 |
| F15 | 4 | 1 | 60 | 49 | 11 |
| F15 | 4 | 2 | 80 | 59 | 56 |
| F15 | 4 | 3 | 90 | 75 | 38 |
| F15 | 5 | 1 | 11 | 7 | 2 |
| F15 | 5 | 2 | 5 | 3 | 0 |
| F15 | 5 | 3 | 14 | 9 | 2 |
| F15 | 6 | 1 | 0 | 0 | 0 |
| F15 | 6 | 2 | 10 | 10 | 9 |
| F15 | 6 | 3 | 26 | 14 | 13 |
| F15 | 7 | 1 | 0 | 0 | 0 |
| F15 | 7 | 2 | 0 | 0 | 0 |
| F15 | 7 | 3 | 0 | 0 | 0 |
| F15 | 8 | 1 | 0 | 0 | 0 |
| F15 | 8 | 2 | 0 | 0 | 0 |
| F15 | 8 | 3 | 17 | 11 | 0 |
| F15 | 9 | 1 | 0 | 0 | 0 |
| F15 | 9 | 2 | 0 | 0 | 0 |
| F15 | 9 | 3 | 11 | 5 | 0 |
| F15 | 10 | 1 | 0 | 0 | 0 |
| F15 | 10 | 2 | 0 | 0 | 0 |
| F15 | 10 | 3 | 0 | 0 | 0 |
| F15 | 11 | 1 | 0 | 0 | 0 |
| F15 | 11 | 2 | 0 | 0 | 0 |
| F15 | 11 | 3 | 42 | 29 | 11 |
| F15 | 12 | 1 | 0 | 0 | 0 |
| F15 | 12 | 2 | 0 | 0 | 0 |
| F15 | 12 | 3 | 50 | 35 | 0 |
| F15 | 13 | 1 | 0 | 0 | 0 |
| F15 | 13 | 2 | 0 | 0 | 0 |
| F15 | 13 | 3 | 0 | 0 | 0 |
| | | | 511 | 358 | 177 |

TABLE 13
Full results achieved by this method.

| | | | Initiation of | | Establishment | | Genotypes which | |
|---|---|---|---|---|---|---|---|---|
| | | | embryogenic | | of embryogenic | | producing mature | |
| | Number of | Number of | tissue | | tissue | | somatic embryos | |
| Family | cones | gametophytes | No. | % | No. | % | No. | % |
| 1 | 8 | 212 | 23 | 10.8 | 6 | 2.8 | 6 | 100 |
| 2 | 12 | 191 | 0 | 0 0 | 0 | 0 0 | 0 | 0 |
| 3 | 7 | 216 | 2 | 0.9 | 1 | 0.5 | 1 | 100 |
| 4 | 7 | 208 | 13 | 6 3 | 9 | 4 3 | 8 | 88 |
| 5 | 7 | 220 | 2 | 0 9 | 0 | 0 0 | 0 | 0 |
| 6 | 10 | 209 | 8 | 3.8 | 3 | 1.4 | 3 | 100 |
| 7 | 8 | 207 | 14 | 6.9 | 10 | 4 8 | 9 | 90 |
| 8 | 8 | 201 | 0 | 0 0 | 0 | 0.0 | 0 | 0 |
| 9 | 16 | 211 | 6 | 2.8 | 0 | 0 0 | 0 | 0 |
| 10 | 6 | 201 | 5 | 2.5 | 2 | 1.0 | 1 | 50 |
| 11 | 8 | 213 | 1 | 0 5 | 0 | 0.0 | 0 | 0 |
| 12 | 8 | 211 | 1 | 0.5 | 0 | 0.0 | 0 | 0 |
| 13 | 8 | 232 | 2 | 0 9 | 1 | 0.5 | 1 | 100 |
| 14 | 6 | 226 | 6 | 2.7 | 6 | 2.7 | 5 | 83 |
| 15 | 6 | 219 | 5 | 2 3 | 0 | 0 0 | 0 | 0 |
| 16 | 8 | 65 | 2 | 3.0 | 0 | 0.0 | 0 | 0 |
| 17 | 9 | 210 | 8 | 3 8 | 5 | 2.4 | 2 | 40 |
| 18 | 10 | 212 | 6 | 2.8 | 3 | 1.4 | 3 | 100 |
| 19 | 6 | 200 | 1 | 0.5 | 0 | 0.0 | 0 | 0 |
| 20 | 5 | 201 | 1 | 0.5 | 0 | 0.0 | 0 | 0 |
| 21 | 8 | 217 | 4 | 1.8 | 1 | 0.5 | 1 | 100 |
| 22 | 7 | 220 | 17 | 7.3 | 0 | 0.0 | 0 | 0 |
| 23 | 11 | 213 | 5 | 2.3 | 1 | 0.5 | 1 | 100 |
| 24 | 6 | 228 | 9 | 4 0 | 8 | 3.5 | 5 | 63 |
| 25 | 8 | 209 | 10 | 4.8 | 4 | 1.9 | 4 | 100 |
| 26 | 8 | 216 | 9 | 4.2 | 0 | 0.0 | 0 | 0 |
| 27 | 8 | 221 | 1 | 0.5 | 0 | 0 0 | 0 | 0 |
| 28 | 7 | 208 | 4 | 1.9 | 0 | 0.0 | 0 | 0 |
| 29 | 6 | 209 | 20 | 9.6 | 14 | 6.7 | 10 | 71 |
| 30 | 6 | 235 | 0 | 0.0 | 0 | 0.0 | 0 | 0 |
| 31 | 7 | 221 | 1 | 0 5 | 1 | 0 5 | 1 | 100 |
| 32 | 6 | 222 | 9 | 4.0 | 3 | 1.4 | 2 | 66 |
| 33 | 20 | 222 | 4 | 1.8 | 0 | 0 0 | 0 | 0 |
| 34 | 8 | 226 | 14 | 6.2 | 10 | 4.4 | 10 | 100 |
| 35 | 6 | 215 | 5 | 2.3 | 4 | 1.9 | 3 | 75 |
| 36 | 8 | 201 | 1 | 0.5 | 1 | 0.5 | 0 | 0 |
| | | 7548 | 220 | 2.9 | 93 | 1.2 | 76 | 82 |

This invention has various important characteristics. As it is the referred modality of the invention, by utilizing different ammonium to nitrate molar ratios simultaneously in order to establish the culture of embryogenic tissue, by transferring the embryogenic masses from the original induction medium to other mediums supplemented with different ammonium to nitrate relation, in such way the continuing proliferation was maintained following the same process for each subculture by transferring small pieces of embryogenic tissue to media with different ammonium to nitrate relation Such different relations used properly give rise mature somatic embryos from a wide range of genotypes, wherein other key was the reduction of proliferation rates of embryogenic tissue previous to apply the maturation pretreatment in medium with medium with very low ammonium to nitrate and the utilization of distilled sterile water to wash the somatic embryos previous to that treatment as well

The result of the invention shown that an ammonium to nitrate relation, specifically low ammonium to nitrate mixed with PEG, ABA and maltose, give rice positively in the maturation of somatic embryos from 82% of produced genotypes belonging to 95% of the trees or families treated.

The application of this invention to conifer trees give rice the regeneration of highest quantity of genotypes per family and families no reported before.

Key Words.

The term “embryogenic tissue” or “embryogenic masses” is the group of immature embryos, which are multiplied or proliferated in constantly growth such translucent and mucilaginous masses that characterizing conifer trees. The term “subculturing” is considered until now as the practice to transfer embryogenic tissue to fresh medium, whereas in this method means to transfer such tissue to different media with proper ammonium to nitrate molar ratio each medium.

### REFERENCES

Aitken-Christie, J and Connett, M. 1992 Micropropagation of Forest Trees. In: Transplant Production System. Kurata, K. y T. Kozai (eds.) Kluwer Academic Publisher, The Netherlands. Pp.: 163-194.

Attree, S 1999 Increasing levels of growth regulator and/or water stress during embryo development. WO9963805 A2 patent. Dec. 16, 1999.

Attree, S. and Fowke, L. C. 1999. Maturation, desiccation and encapsulation of gymnosperm somatic embryos U.S. Pat. No. 5,985,667. Nov. 16, 1999

Attree, S. M. and Fowke, L C. 1993. Embryogeny of gymnosperms: advances in synthetic seed technology of conifer. Plant Cell Tiss. Org. Cult. 35: 1-35

Attree, S M.; Dunstan, D. I. and Fowke, L. C. 1989. Plant regeneration from embryogenic protoplasts of white spruce (Picea glauca) Bio/Technol 7. 1060-1062.

Attree, S. M.; Pomeroy, M. K. and Fowke, L. C. 1994 Production of vigorous, desiccation, tolerant white spruce (Picea glauca (Moench,) Voss.) synthetic seed m bioreactor. Plant Cell Rep. 13: 601-606.

Becwar, M R; Chesick, E. E.; Handley, III, L. W. and Rutter M. R. 1995. Method for regeneration of coniferous plants by somatic embryogenesis. U.S. Pat. No. 5,413,930. May 9, 1995.

Coke, J. E. 1996. Basal nutrient medium for in vitro cultures of loblolly pines. U.S. Pat. No. 5,534,434. Jul. 9, 1996.

DeVerno, L. L.; Park, Y. S.; Bonga, J. M, Barret, J. D. and Simmpson. C. 1999. Somaclonal variation in cryopreserved embryogenic clones of white spruce (Picea glauca (Moench) Voss.). Plant Cell Rep. 18 (11): 948-953.

Dunstan, D. I.; Bethune, T. D. and Bock, C. A. 1993. Somatic embryo maturation from long-term suspension cultures of white spruce (Picea glauca).In vitro Cell. Dev. Biol. 29.109-112

Eastman, P. A. K.; Webster, F. B; Pitel, J. A. and Roberts, D. R. 1991. Evaluation of somaclonal variation during somatic embryogenesis of interior spruce (Picea glauca engelmanncomplex) using culture morphology and isozyme analysis. Plant Cell Rep. 10: 425-430.

Finer, J J; Kriebel, H. B. and Becwar, M. R. 1989. Initiation of embryogenic callus and suspension cultures of eastern white pine (Pinus strobus L.) Plant Cell Reports 8: 203-206

Garin, E; Isabel, N. and Plourde, A 1998. Screening of large numbers of seed families of Pinus strobus L. For somatic embryogenesis from immature and mature zygotic embryos. Plant Cell Rep. 18: 37-43.

George, E F 1993 The Technology. In: Plant propagation by tissue culture. Partl. Exegetics Ltd (eds.) Butler & Tanner Ltd. England. Pp. 470.

Gupta, P. K. 1996. Method for reproducing conifers by somatic embryogenesis using a maltose enriched maintenance medium. U.S. Pat. No. 5,563,061. Oct. 8, 1996.

Gupta, P. K. and Durzan, D. J. 1987a Biotechnology of somatic polyembryogenesis and plantlet regeneration in loblolly pine. Bio/Technol. 5: 147-151

Gupta, P. K. and Durzan, D. J. 1987b. Somatic embryos from protoplast of loblolly pine proembryonal cells. Bio/Technol. 5: 710-712.

Gupta, P. K and G. S. Pullman. 1991. High concentration enrichment of conifer embryonal cells. U.S. Pat. No. 5,041,382. Aug. 20, 1991.

Gupta, P. K. and G. S. Pullman. 1991. Method for reproducing conifers by somatic embryogenesis using abscisic acid and osmotic potential variation. U.S. Pat. No. 5,036,007. Jul. 30, 1991.

Gupta, P. K. and G. S. Pullman. 1993 Method for reproducing conifers by somatic embryogenesis using stepwise hormone adjustment. U.S. Pat. No. 5,236,841. Aug. 17, 1993.

Gupta, P K.; Pullman, G.; Timmis, R.; Kreitinger, M; Carlson, W. C.; Grob, J and Welty, E. 1993 Forestry in the 21^{st}. century The biotechnology of somatic embryogenesis. Bio/Technol. 11: 454-459.

Hakman, I and von Arnold, S 1985. Plantlet regeneration through somatic embyogenesis in Picea abies (Norway spruce) J Plant Physiol 121: 579-587.

Handley III, L W. and Godbey, A. P. 1996. Embryogenic coniferous liquid suspension cultures. U.S. Pat. No. 54491090. Feb. 13, 1996.

Handley, III, L. W. 1998. Method for regeneration of coniferous plants by somatic embryogenesis. U.S. Pat. No. 5,731,203. Mar. 24, 1998.

Handley, III, L. W. 1999 Method for regeneration of coniferous plants by somatic embryogenesis in culture media containing abscisic acid. U.S. Pat. No. 5,856,191. Jan. 5, 1999.

Hogberg, K -A.; Ekberg, I.; Norell, L. and von Arnold, S. 1998. Integration of somatic embryogenesis in a tree breeding programme: a case study with Picea abies. Can. J. For. Res 28 1536-1545

Joy, R. W.; Parkash, P. K. and Thorpe, T. A. 1991. Long term storage of somatic embryogenic white spruce tissue at ambient temperature. Plant Cell Tiss. Org. Cult. 25: 53-60.

Klimaszewska, K. and Smith, D. 1997. Maturation of somatic embryos of Pinus strobus is promoted by high concentration of gellan gum. Physiol Plant. 100: 949-957.

Laine, E. and David, A. 1990. Somatic embryogenesis in immature embryos and protoplasts of Pinus caribaea. Plant Science 69: 215-224

Laine, E.; Bade, P. and David, A. 1992. Recovery of plants from cryopreserved embryogenic cell suspensions of Pinus caribaea. Plant Cell Rep. 11: 295-298.

Lelu, M. A.; Bastien, C, Drugeault, A Gouez, M. L and Klimaszewska, K. 1999. Somatic embryogenesis and plantlet development in Pinus sylvesiris and Pinus pinaster on medium with and without growth regulators. Physiol Plant 105-719-728.

McVaugh, R. 1992. Gymnosperms and Pteridophytes. In: Flora Novogaliciana Anderson, W. R. (ed.) University of Michigan United States of America Vol. 17: 1-119.

Pullman, G S. and P K. Gupta 1991. Method for reproducing coniferous plants by somatic embryogenesis using adsorbent materials in the development stage media. U.S. Pat. No. 5,034,326. Jul. 23, 1991.

Pullman, G. S and P. K. Gupta. 1994. Method for reproducing conifers by somatic embryogenesis using mixed growth hormones for embryo culture. U.S. Pat. No. 5,294,549. Mar. 15, 1994.

Ramírez-Serrano, C. 2000. Conservación en refrigeración, cultivo en suspension y maduración de embriones somáticos de gimnospermas. Filled Patent MX001185. Feb. 3, 2000.

Ramírez-Serrano, C. 1996. Embriogénesis somática en pino piñonero (Pinus maximartinezii Rsedowski). MS Thesis. CIATEJ-UDEG. Guadalajara Jal Mexico. 72 pp.

Ramírez-Serrano, C., Bozkhob, P.; Ekberg, I and von Arnold, S. 1999a Potential of somatic embryogenesis for a wide range of genotypes in Scots pine (Pinus sylvestris L.). In: Program-Abstracts XIX Congress Scandinavian Society of Plant Physiology. Joensuu Finland, Jun. 21-23, 1999. Pp. 103.

Ramírez-Serrano, C.; Bozkhob, P.; Ekberg, I. and von Arnold, S 1999b. Inter and Intra family effects in somatic embryogenesis of scots pine In: Abstracts Forest Biotechnology '99. A join Meeting of: The International Wood Biotechnology Symposium and IUFRO Working Party 2.40-06 Molecular Genetics of Trees. Kable College, University of Oxford, Oxford, United Kingdom Jul. 11-16, 1999. Poster 7.

Rutter, M. R.; Handley, III, L. W. and M R Becwar. 1998, Method for regeneration of coniferous plants by somatic embryogenesis employing polyethilene glycol. U.S. Pat. No. 5,731,204. Mar. 24, 1998

Tautorus, T. E.; Fowke, L. C. and Dunstan, D L 1991 Somatic embryogenesis in conifers. Can. J. Bot. 69-1873-1899.

Uddin, M. R. 1993. Somatic embryogenesis in gymnosperms. U.S. Pat. No. 5,187,092. Feb. 16,1993.

von Arnold, S. 1987. Improved efficiency of somatic embryogenesis in mature embryos of Picea abies (L.) Karst. J. Plant Physiol. 128: 233-244.

Webster, F. B.; Roberts, D. R.; McInnis, S. M. and Sutton, B. C. S. 1990. Propagation of interior spruce by somatic embryogenesis. Can. J. For, Res. 20:1759-1765.

1. A method for producing mature somatic embryos from a wide range of cryopreserved or no cryopreserved genotypes belonging to different families, by using solid medium in all the steps of the process This method comprises the establishment of embryogenic tissue, continuing proliferation, the treatment to reduce the proliferation, the treatment to initiate the immature somatic embryo development, and the somatic embryo maturation

2. A method in accordance with claim 1, wherein said embryogenic tissue is the embryo masses which are translucent and mucilaginous, such masses are subcultured in different mediums

3. A method in accordance with claim 2, wherein said embryogenic tissue is established and proliferates for at least 12 months in which the subculture is done among 1 and 4 weeks by exchanging embryogenic masses among different media.

4. A method in accordance with claim 3, wherein different media are used, each one with different ammonium to nitrate molar ratio, such relation can be between low ammonium to nitrate to high ammonium to nitrate, plus carbon source and plant growth regulators.

5. A method in accordance with claim 4, wherein the ammonium to nitrate molar ratio can be in the range from 01:99 to 99 01.

6. A method in accordance with claim 4, wherein the carbon sources is sucrose, maltose, or fructose.

7. A method in accordance with claim 4, wherein the sucrose, maltose or fructose concentration is in the range from 1 to 6%.

8. A method in accordance with claim 4, wherein the plant growth regulators concentration on medium is in a relation of 2,4-Diclorofenoxiacetic acid and Benziladenine varying between 10 2 to 0 1:0.01.

9. A method in accordance with claim 4, wherein the plant growth regulators concentration is varying from 0.1 to 10 mg/l 2,4-Diclorofenoxiacetico acid and from 0.01 to 2 mg/l Benziladenine

10. A method in accordance with claim 1, wherein the treatment to reduce proliferation rates is by subculturing in a medium with low ammonium to nitrate molar ratio, a carbon source, for a period from 1 to 45 days prior to start of immature somatic embryos maturation treatment.

11. A method in accordance with claim 10, wherein the low ammonium to nitrate molar ratio is ranges from 01:99 to 40:60.

12. A method in accordance with claim 10, wherein the carbon source is either sucrose or maltose.

13. A method in accordance with claim 10, wherein the carbon source concentration ranges between 1 to 6%

14. A method in accordance with claim 1, wherein the treatment for developing the immature somatic embryos is subculturing in medium with low ammonium to nitrate molar ratio, a chemical adsorbent, a carbon source and lacking of plant growth regulators, for a period ranging from 1 to 12 weeks

15. A method in accordance with claim 14, wherein the low ammonium to nitrate molar ratio varying in a range from 0.1:99 to 40:60

16. A method in accordance with claim 14, wherein said chemical adsorbent is activated charcoal

17. A method in accordance with claim 16, wherein the concentration of activated charcoal is ranges between 0.5 to 2 0%

18. A method in accordance with claim 14, wherein said carbon source is maltose.

19. A method in accordance with claim 18, wherein said maltose is present in the medium in a concentration that ranges between 1 a 10%.

20. A method in accordance with claim 14, wherein said treatment to initiate somatic embryo development included to wash between 1 to 5 times the embryogenic tissue with distilled sterile water previous to spread on filter paper which is over the medium with chemical adsorbent.

21. A method in accordance with claim 14, wherein said treatment to initiate somatic embryo development included to transfer onto filter paper between 100 to 1000 mg of embryogenic tissue such thin layer without accumulation of embryo masses.

22. A method according with claim 1, wherein said maturation of somatic embryos is the developmental process of embryo head until cotyledonary embryo on maturation medium.

23. A method according with claim 1, wherein said somatic embryo maturation to the embryos originated from cryopreserved genotypes or no cryopreserved genotypes.

24. A gymnosperm mature somatic embryo characterized by giving the ancestral immature somatic embryo a establishment proliferation treatment through subculturing at least in two media, a high quality proliferation at least in two media, a reduction of proliferation rate previous to maturation treatment according with the genotype used, having a treatment to start the development of somatic embryo and having maturation treatment by using a medium supplemented with a low ammonium to nitrate molar ratio, a carbon source, a maturation promoter, and a desiccant compound.

25. A method according with claim 24, wherein said low ammonium to nitrate relation is ranging from 0 1 99 to 40 60

26. A method in accordance with claim 24, where said carbon source is maltose

27. A method in accordance with claim 24, wherein maltose concentration is ranging from 1% to 10%

28. A method according with claim 24, wherein said a maturation promoter is ABA or analogous.

29. A method according with claim 24, wherein ABA concentration onto named medium has a range between 20 a 120 μM.

30. A method in accordance with claim 24, wherein said a desiccant compound onto the medium is PEG 4000

31. A method in accordance with claim 24, wherein the PEG4000 concentration is ranging between 1 to 10%.

32. A gymnosperm mature somatic embryo, in accordance with claim 24, characterized by being analogous to gymnosperm zygotic embryo.

33. A gymnosperm mature somatic embryo, in accordance with claim 32, wherein said gymnosperm is a conifer.

34. A conifer mature somatic embryo, in accordance with claim 33, wherein said mature somatic embryo is from the family Pinaceae.

35. A conifer mature somatic embryo, in accordance with claim 34, wherein said mature somatic embryo is from the genus Pinus.

36. A pinacea mature somatic embryo, in accordance with claim 34, wherein said embryo is every one from the genus Pinus.

37. A method in accordance with claim 23, wherein said embryos are maturated in a time varying from 1 to 14 weeks.

38. A method in accordance with claim 23, wherein said embryos are maturated in a time varying from 3 to 10 weeks.

39. A method in accordance with claim 23, wherein said embryos are maturated in a time varying from 5 to 8 weeks.
